(19) United States
(12) Patent Application Publication
(10) Pub. No.: US 2024/0216567 A1
(43) Pub. Date: Jul. 4, 2024

(54) PHOTOCATALYST DEVICE

(71) Applicant: Hanon Systems, Daejeon (KR)

(72) Inventors: Chang Hyun BAEK, Daejeon (KR); Young Chul KIM, Daejeon (KR); Sang Ho OH, Daejeon (KR); Chae Geun LIM, Daejeon (KR); Yun Sung JO, Daejeon (KR)

(21) Appl. No.: 18/562,883

(22) PCT Filed: Jun. 8, 2022
(86) PCT No.: PCT/KR2022/008074

§ 371 Date: Nov. 21, 2023

(30) Foreign Application Priority Data
Jun. 30, 2021 (KR) 10-2021-0085386
May 31, 2022 (KR) 10-2022-0066777

(51) Int. Cl. A61L 9/18; B01D 53/00; B01D 53/88; B60H 3/06; G02B 3/00; G02B 5/02; G02B 19/00

(52) U.S. Cl. CPC A61L 9/18; B01D 53/007; B01D 53/885; B60H 3/06; G02B 3/0056; G02B 5/0215; G02B 5/0278; G02B 5/0294; G02B 19/0009; G02B 19/0047; A61L 2209/12; A61L 2209/16; B01D 2255/802; B01D 2257/90; B01D 2257/91; B01D 2259/4566; B01D 2259/802; B60H 2003/0675

(57) ABSTRACT

A photocatalyst device reducing the separation distance between a light source and a catalyst part while securing visible light illumination over the entire range of the catalyst part, reducing the package size, and increasing the amount of visible light irradiated to the catalyst part to enhance its photocatalytic reactivity.

The photocatalyst device, generating a photocatalytic reaction and emitting superoxide radicals into internal flow path of air conditioning case to purify air blown into a vehicle passenger room along the internal flow path, includes a photocatalyst body with a light source part and a catalyst part installed on it, where catalyst part causes a photocatalytic reaction by light emitted from the light source part, and a light processing part processing the light emitted from the light source part so as to increase an irradiance rate of the light of the light source part with respect to the catalyst part.

### TECHNICAL FIELD

The present invention relates to a photocatalyst device, and more particularly, a photocatalyst device configured to reduce the separation distance between a light source and a catalyst part while securing visible light illumination over the entire range of the catalyst part and, so that the package can be reduced to achieve miniaturization and slimming, and configured to appropriately process the visible light of the light source, so that the amount of visible light irradiated to the catalyst part can be increased to enhance photocatalytic reactivity in the catalyst part.

### BACKGROUND ART

Motor vehicles are equipped with an air conditioning system that adjusts the temperature inside a passenger room. The air conditioning system sucks in air from inside and outside the passenger room, heats or cools the sucked air to an optimal temperature, and blows the air into the passenger room.

Specifically, in winter, the air to be blown into the passenger room is heated to keep the passenger room warm, and in summer, the air to be blown into the passenger room is cooled to keep the passenger room cool. Therefore, the temperature inside the passenger room can be always maintained in a comfortable state.

In such an air conditioning system, it is an important task to maintain the air temperature and the air quality inside the passenger room at an optimal state.

In particular, the air inside the passenger room is easily contaminated due to the narrow and closed environment and the existence of fine dust and various contaminants introduced from the outside. It is an important task to maintain the air quality inside the passenger room at an optimal level in response to such air contamination in the passenger room.

As a method for improving air quality inside a passenger room, there is a technique that makes use of a photocatalyst device.

In this technique, as shown in FIG. 1, a photocatalyst device 1 equipped with a light source part 3 and a catalyst part 5 is installed on an internal flow path 7a of an air conditioning case. The light source part irradiates visible light to the catalyst part 5 to induce a photocatalytic reaction in the catalyst part 5. Superoxide radicals are generated through the photocatalytic reaction in the catalyst part 5. The bad odors, bacteria, and contaminants in the air blown into the passenger room are removed by the superoxide radicals.

Typically, the light source part 3 uses an LED lamp as a light source. The catalyst part 5 is composed of a metal foam substrate with a three-dimensional network structure and a photocatalyst coating layer coated on the surface of the metal foam substrate.

Since the photocatalyst device 1 removes bad odor, bacteria, and contaminants in the air blown into the passenger room, it is possible for the photocatalyst device 1 to improve the cleanliness of the air blown into the passenger room, thereby improving the air quality inside the passenger room.

This conventional photocatalyst device 1 has a structure in which a sufficient separation distance L is secured between the light source part 3 and the catalyst part 5 in order to irradiate a sufficient amount of visible light to the entire catalyst part 5. Therefore, there is a limit in downsizing a package.

Specifically, in the case of the light source part 3, an LED lamp having a light irradiation range α of about 120° is used as a light source. In order to irradiate visible light to the entire catalyst part 5 in conformity with the light irradiation range a, a minimum separation distance L between the light source part 3 and the catalyst part 5 is calculated through equation (1) represented below, and then the separation distance between the light source part 3 and the catalyst part 5 equal to or larger than the minimum separation distance L has to be secured.

Separation distance (L)=catalyst part width (W)/2×tan(60) (1)

Thus, in view of the interference with surrounding components and the need for miniaturization and slimming to increase space utilization, there is a limit in reducing the package of the photocatalyst device 1.

Additionally, the conventional photocatalyst device 1 has a disadvantage in that the visible light from the light source part 3 cannot be sufficiently diffused into the catalyst part 5 due to the metal foam substrate of the catalyst part 5.

In particular, the metal foam substrate having a three-dimensional network structure impedes the diffusion of visible light irradiated from the light source part 3. Because of this impediment of the diffusion of visible light, the irradiance rate of visible light decreases significantly toward the inside of the catalyst part 5.

Therefore, the photocatalytic reactivity in the catalyst part 5 is low, and the amount of superoxide radicals generated in the catalyst part 5 is reduced, which reduces the air purification efficiency in the passenger room.

### SUMMARY

In view of the problems inherent in the related art, it is an object of the present invention to provide a photocatalyst device capable of reducing the distance between a light source and a catalyst part while achieving visible light irradiation to the entire catalyst part.

Another object of the present invention is to provide a photocatalyst device capable of reducing a package and consequently achieving miniaturization and slimming by adopting a configuration in which the distance between a light source and a catalyst part can be reduced while achieving visible light irradiation to the entire catalyst part.

A further object of the present invention is to provide a photocatalyst device capable of irradiating sufficient visible light to the entire catalyst part regardless of the locations.

A further object of the present invention is to provide a photocatalyst device capable of enhancing the photocatalytic reactivity of the entire catalyst part, increasing the amount of superoxide radicals generated in a catalyst part, and improving the air purification efficiency in a passenger room.

In order to achieve these objects, there is provided a photocatalyst device for generating a photocatalytic reaction and emitting superoxide radicals into an internal flow path of an air conditioning case to purify air blown into a passenger room of a vehicle along the internal flow path, the photocatalyst device comprising: a photocatalyst body; a light source part installed on the photocatalyst body; a catalyst part installed on the photocatalyst body so as to be spaced apart from the light source part and configured to cause a photocatalytic reaction by light emitted from the light source part; and a light processing part configured to process the light emitted from the light source part so as to increase an irradiance rate of the light of the light source part with respect to the catalyst part.

The light processing part includes a light diffusion part configured to diffuse the light emitted from the light source part to the catalyst part so as to expand a light irradiation range of the light source part with respect to the catalyst part, and a light straightening part configured to improve straight movement of the light irradiated from the light source part to the catalyst part so as to increase the depth of light irradiation of the light source part with respect to the catalyst part.

The light source part includes a plurality of light sources which are LED lamps.

The light sources include a central light source arranged at center and peripheral light sources arranged around the central light source at predetermined intervals.

The light diffusion part includes light diffusion lenses installed on light exit portions of one or more light sources, and the light diffusion lens are configured to diffuse the light emitted from the light sources toward the catalyst part.

The light diffusion lenses are installed to correspond to the peripheral light sources arranged around the central light source, and are configured to diffuse the light emitted from the peripheral light sources toward the catalyst part.

The number of the light diffusion lenses is adjusted depending on the area of the catalyst part.

The number of the light diffusion lenses is determined by equation (2) represented below:

number of light diffusion lenses (N)={π×(width of catalyst part (W)/2)²}/π×(separation distance between catalyst part and light source (L)×tan(90−light irradiation angle of light source enlarged by light diffusion lens/2))² (2)

The light straightening part includes a light concentrating lens installed on a light exit portion of at least one of the light sources, and the light concentrating lens is configured to concentrate the light emitted from the light sources toward the catalyst part to improve the straight movement of the light toward the catalyst part.

The light concentrating lens is installed to correspond to the central light source among the plurality of light sources, and is configured to concentrate the light emitted from the central light source on the catalyst part.

The light processing part includes a fly eye lens installed between the light exist portion of the light source part and the catalyst part to improve homogeneity of the light irradiated from the light source part to the catalyst part.

The fly eye lens includes a lens body sheet installed between the light source part and the catalyst part, and a plurality of lens cells formed at intervals on the surface of the lens body sheet to improve light homogeneity by repeatedly overlapping the light emitted from the light source part to the catalyst part.

The photocatalyst device further comprises: reflection members configured to reflect the light inside the catalyst part transmitted outward through a side portion of the catalyst part toward the catalyst part.

According to the photocatalyst device of the present invention, visible light is irradiated to the catalyst part by a plurality of light sources. Therefore, it is possible to significantly increase the amount of visible light irradiated to the catalyst part.

In addition, since the amount of visible light irradiated to the catalyst part can be significantly increased, it is possible to enhance the photocatalytic reactivity in the catalyst part, thereby generating a large amount of superoxide radicals. As a result, it is possible to improve the purification efficiency for the air blown into the passenger room and improve the cleanliness of the air in the passenger room.

In addition, since the visible light irradiated from the light source to the catalyst part is diffused through the light diffusion part, it is possible to significantly expand the visible light irradiation range of the light source with respect to the catalyst part.

In addition, since the visible light irradiation range of the light source with respect to the catalyst part can be significantly expanded, it is possible to shorten the separation distance between the catalyst part and the light source under condition that the entire catalyst part can be irradiated. As a result, it is possible to reduce the separation distance between the catalyst part and the light source while sufficiently securing the visible light irradiation to the entire catalyst part.

In addition, since the separation distance between the catalyst part and the light source can be reduced while sufficiently securing the visible light irradiation to the entire catalyst part, it is possible to downsize the package without deteriorating the photocatalytic reaction in the catalyst part, thereby miniaturizing and sliming the device.

In addition, since the straight movement of visible light irradiated from the light source to the catalyst part can be improved through the light straightening part, it is possible to allow the visible light from the light source to reach the inside of the catalyst part and the portion opposite to the light source.

In addition, since the visible light from the light source can be allowed to reach the inside of the catalyst part and the portion opposite to the light source, it is possible to allow sufficient visible light to be irradiated to the entire catalyst part regardless of the locations. As a result, it is possible to enhance the photocatalytic reactivity in the catalyst part and improve the air purification efficiency in the passenger room.

In addition, since the visible light passing through the side portion of the catalyst part is reflected back to the inside of the catalyst part through the reflection member, it is possible to increase the amount of visible light irradiated to the catalyst part. As a result, it is possible to enhance the photocatalytic reactivity in the catalyst part and improve the air purification efficiency in the passenger room.

### BRIEF DESCRIPTION OF THE DRAWINGS

FIG. 1 is a sectional view showing a conventional photocatalyst device.

FIG. 2 is an exploded perspective view showing the configuration of a photocatalyst device according to a first embodiment of the present invention.

FIG. 3 is a perspective view showing the assembled state of the photocatalyst device according to the first embodiment of the present invention shown in FIG. 2.

FIG. 4 is a side sectional view of the photocatalyst device shown in FIG. 3.

FIG. 5 is an enlarged sectional view showing the main parts of the photocatalyst device according to the first embodiment of the present invention.

FIG. 6 is an exploded perspective view showing the configuration of a photocatalyst device according to a second embodiment of the present invention.

FIG. 7 is a perspective view showing the assembled state of the photocatalyst device according to the second embodiment of the present invention shown in FIG. 6.

FIG. 8 is a side sectional view of the photocatalyst device shown in FIG. 7.

FIGS. 9A and 9B are graphs for comparing the operating performance of the photocatalyst device according to the present invention with that of the prior art. FIG. 9A is a three-dimensional luminance map graph showing the amount of light absorbed by the catalyst part of the photocatalyst device of the present invention, and FIG. 9B is a three-dimensional luminance map graph showing the amount of light absorbed by a catalyst part of a conventional photocatalyst device.

### DETAILED DESCRIPTION

Preferred embodiments of a photocatalyst device according to the present invention will now be described in detail with reference to the accompanying drawings.

### First Embodiment

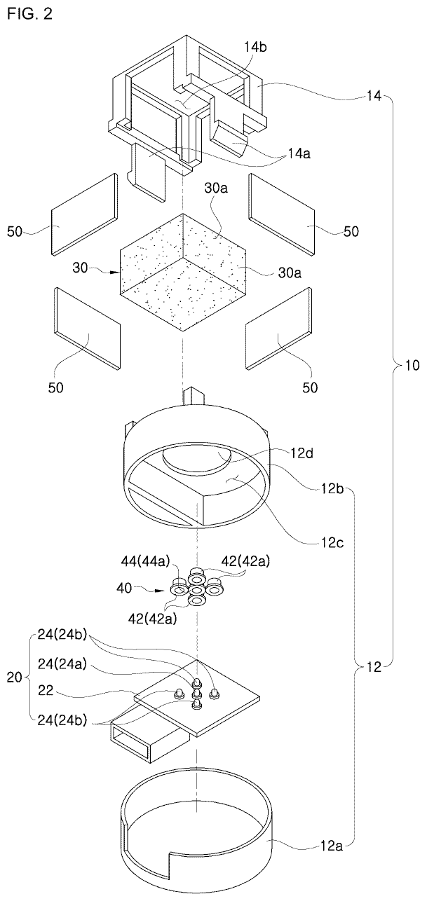

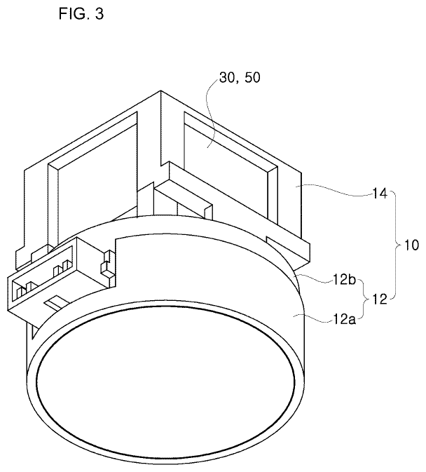

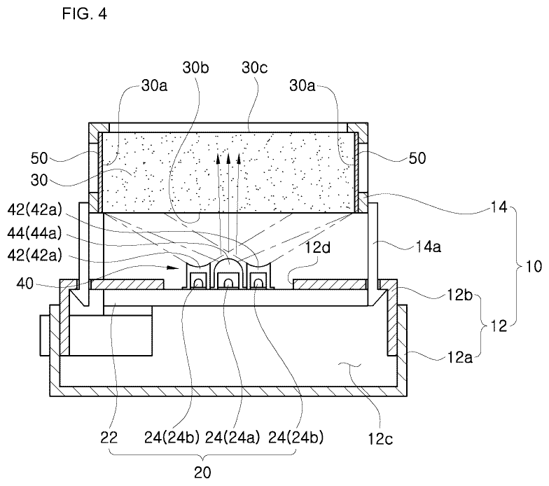

Referring to FIGS. 2 to 4, the photocatalyst device according to a first embodiment of the present invention includes a photocatalyst body 10, a light source part 20, and a catalyst part 30. The light source part 20 and the catalyst part 30 are installed on the photocatalyst body 10 to correspond to each other.

The photocatalyst body 10 includes a first body part 12 on which the light source part 20 is installed, and a second body part 14 on which the catalyst part 30 is installed.

The first body part 12 has a cylindrical shape and is composed of a first case 12a and a second case 12b fitted to each other. The first case 12a and the second case 12b fitted to each other defines an internal installation space 12c.

The second body part 14 is fixedly installed on one side of the first body part 12 through hooks 14a and is equipped with a catalyst accommodation portion 14b. The catalyst accommodation portion 14b is configured to accommodate the catalyst part 30 and is opened on all sides.

Referring again to FIGS. 2 to 4, the light source part 20 is installed in the installation space 12c of the first body part 12.

The light source part 20 includes a PCB panel 22 and a light source 24 installed on the PCB panel 22.

The light source part 20 is installed in the internal installation space 12c of the first body part 12. In particular, the light source part 20 is installed such that the light source 24 can face the catalyst part 30 accommodated in the catalyst accommodation portion 14b of the second body part 14.

The first body part 12 has a through hole 12d through which the light source 24 of the light source part 20 installed in the internal installation space 12c can pass. The light source 24 of the light source part 20 can face the catalyst part 30 of the second body part 14.

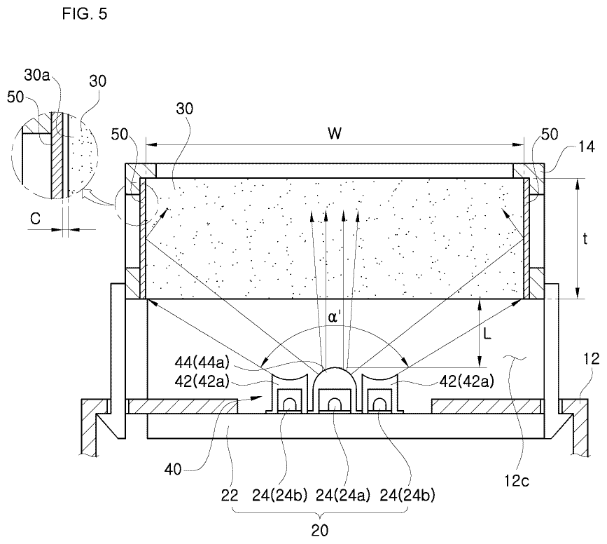

The catalyst part 30 of the second body part 14 is composed of a hexahedron. As shown in FIG. 5, the catalyst part 30 is installed with a predetermined separation distance L from the light source 24 of the light source part 20 installed in the first body part 12.

The catalyst part 30 generates superoxide radicals while causing a photocatalytic reaction by the visible light irradiated from the light source 24 of the light source part 20.

Therefore, the generated superoxide radicals remove bad odor, bacteria, and various contaminants in the air blown into the passenger room. As a result, the air quality inside the passenger room can be improved by increasing the cleanliness of the air blown into the passenger room.

Referring again to FIGS. 2 to 4, the photocatalyst device of the present invention includes a light source part 20, wherein the light source part 20 includes a plurality of light sources 24.

Each of the light sources 24 is composed of LED lamps. The light sources 24 are installed at intervals in a portion of the PCB panel 22.

In particular, the light sources 24 are installed in the portion of the PCB panel 22 corresponding to the central portion of the catalyst part 30.

Preferably, the light sources 24 are installed in a portion of the PCB panel 22, so that one light source 24 (hereinafter referred to as “central light source 24a”) is arranged at the center and other light sources 24 (hereinafter referred to as “peripheral light sources 24b”) are arranged at regular intervals along the periphery of the central light source 24a.

In particular, the peripheral light sources 24b are preferably arranged at regular intervals on a concentric circle centered on the central light source 24a.

In some cases, the light sources 24 may be arranged in rows and columns on a portion of the PCB panel 22.

Preferably, four peripheral light sources 24b are arranged along the periphery of the central light source 24a.

The light sources 24 are operated by electric power to irradiate visible light to the catalyst part 30.

Therefore, the amount of light irradiated to the catalyst part 30 is significantly increased. This makes it possible to improve the photocatalytic reaction in the catalyst part 30 and to consequently generate a large amount of superoxide radicals.

Referring again to FIGS. 2 to 4, the photocatalyst device of the present invention includes a light processing part 40 configured to process visible light emitted from the light source part 20 so as to increase the visible light irradiance rate of the light source part 20 with respect to the catalyst part 30.

The light processing part 40 includes a light diffusion part 42 for diffusing the visible light irradiated from the light source part 20 to the catalyst part 30, and a light straightening part 44 for improving the straight movement of the visible light irradiated from the light source part 20 to the catalyst part 30.

The light diffusion part 42 is composed of a light diffusion lens 42a installed on the light exit portion of at least one of the light sources 24.

The light diffusion lens 42a is composed of plano-concave lenses installed at the front light exit portions of the peripheral light sources 24b around the central light source 24a among the plurality of light sources 24.

At this time, the plano-concave lenses are installed so that the planar portions thereof face the peripheral light sources 24b side and the concave portions thereof face the catalyst part 30 side.

As shown in FIG. 5, this light diffusion lens 42a serves to diffuse the visible light emitted from the peripheral light sources 24b.

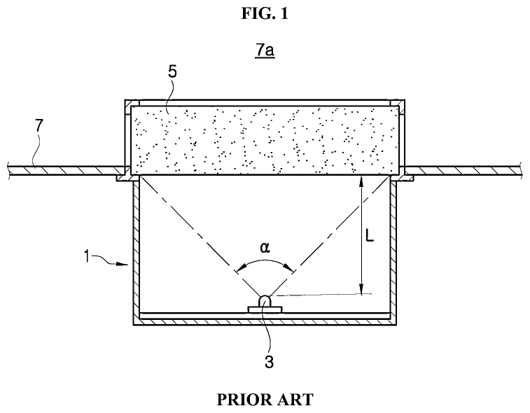

In particular, the visible light emitted from the light sources 24 over an irradiation range α of about 120° (see FIG. 1) is diffused to have an irradiation angle α′ larger than 120°.

Therefore, it is possible to significantly expand the visible light irradiation range α′ of the light source 24 to the catalyst part 30.

Thus, under condition that the entire catalyst part 30 can be irradiated, it is possible to create room to shorten the separation distance L between the catalyst part 30 and the light sources 24.

As a result, it is possible to reduce the separation distance L between the catalyst part 30 and the light sources 24 while sufficiently securing the visible light irradiation to the entire catalyst part 30.

Accordingly, it is possible to downsize the package without deteriorating the photocatalytic reaction in the catalyst part 30, thereby miniaturizing and sliming the device.

Meanwhile, the number of light diffusion lenses 42a may be adjusted depending on the size of the catalyst part 30. For example, as the area of the catalyst part 30 to which the visible light is irradiated, that is, the width W of the catalyst part 30, becomes larger, the number of light diffusion lenses 42a can be increased proportionally.

Preferably, the number of light diffusion lenses 42a may be determined by equation (2) represented below.

Number of light diffusion lenses (N)={π×(width of catalyst part (W)/2)²}/π×(separation distance between catalyst part and light source (L)×tan(90−light irradiation angle of light source enlarged by light diffusion lens/2))² (2)

Referring again to FIGS. 2 to 4, the light straightening part 44 is composed of a light concentrating lens 44a installed on the light exit portion of at least one of the light sources 24.

The light concentrating lens 44a is composed of a plano-convex lens installed on the front light exit portion of the central light source 24a among the plurality of light sources 24. The plano-convex lens is installed such that the planar portion thereof faces the central light source 24a and the convex portion thereof faces the catalyst part 30.

As shown in FIG. 5, the light concentrating lens 44a serves to concentrate the visible light emitted from the central light source 24a.

In particular, the light concentrating lens 44a improves the straight movement of visible light to the catalyst part 30 by concentrating the visible light emitted from the central light source 24a,

Therefore, the visible light irradiation depth of the light source 24 with respect to the catalyst part 30 is increased. This allows the visible light of the light sources 24 to reach the inside of the catalyst part 30 and the portion opposite to the light sources 24.

In particular, by improving the straight movement of visible light, the visible light of the light sources 24 can be irradiated to the inside of the catalyst part 30 and the portion opposite to the light sources 24, despite the impediment of diffusion of visible light by the metal foam substrate of the catalyst part 30.

Thus, sufficient visible light can be irradiated to the entire catalyst part 30, regardless of the locations of individual portions such as the front surface portion, the back surface portion, the inner portion and the outer portion of the catalyst part 30. As a result, the photocatalytic reactivity in the catalyst part 30 can be improved by increasing the amount of visible light irradiated to the entire catalyst part 30.

Accordingly, the air purification efficiency in the passenger room can be significantly improved by increasing the amount of superoxide radicals generated in the catalyst part 30.

Meanwhile, it is desirable to install the light concentrating lens 44a only when the thickness of the metal foam substrate of the catalyst part 30 exceeds a preset thickness.

For example, it is preferable to install the light concentrating lens 44a only under the condition that the thickness t of the metal foam substrate of the catalyst part 30 exceeds 5 mm.

The reason for adopting this configuration is that, when the thickness of the metal foam substrate of the catalyst part 30 is as thin as 5 mm or less, the visible light emitted from the light sources 24 can reach the inside of the catalyst part 30 and the portion opposite to the light sources 24 even without the light concentrating lens 44a.

In some cases, a plurality of light concentrating lenses 44a may be installed so as to correspond to the plurality of light sources 24.

In particular, the number of light concentrating lenses 44a can be adjusted depending on the size of the catalyst part 30. For example, as the area of the catalyst part 30 to which the visible light is irradiated, that is, the width W of the catalyst part 30, becomes large, the number of light concentrating lenses 44a can be increased proportionally.

Referring again to FIGS. 2 to 4, the photocatalyst device of the present invention further includes reflection members 50 installed around the side surface of the catalyst part 30.

The reflection members 50 are plates coated with a light reflecting material and is installed on the side portion 30a of the catalyst part 30.

In particular, the reflection members 50 are installed on all the side portions 30a of the catalyst part 30 except the rear portion 30b on which the visible light emitted from the light sources 24 is incident, and the front portion 30c from which superoxide radicals are emitted.

As shown in FIG. 5, the reflection members 50 serve to reflect the visible light toward the inside of the catalyst part 30 when the visible light of the light sources 24 that has passed through the inside of the catalyst part 30 moves to the outside through the side portion 30a of the catalyst part 30.

Accordingly, the amount of visible light irradiated to the catalyst part 30 can be increased. As a result, it is possible to improve the photocatalytic reaction in the catalyst part 30 so as to generate a large amount of superoxide radicals.

Meanwhile, the reflection members 50 are preferably installed on the side portion 30a of the catalyst part 30 at a predetermined gap c with respect to the side portion of the catalyst part 30. For example, it is preferable to install the reflection members 50 at a gap c of about 3 mm with respect to the side portion of the catalyst part 30.

This is to increase the light reflection efficiency of the reflection members 50 and reflect as much visible light as possible into the inside of the catalyst part 30.

### Second Embodiment

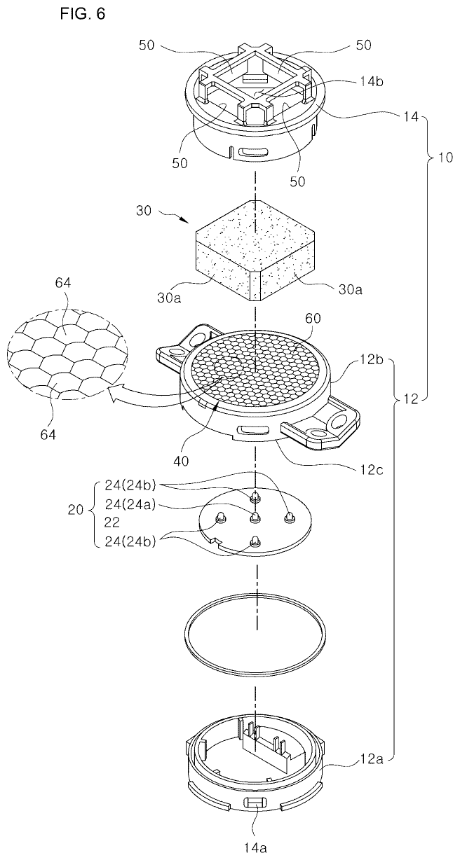

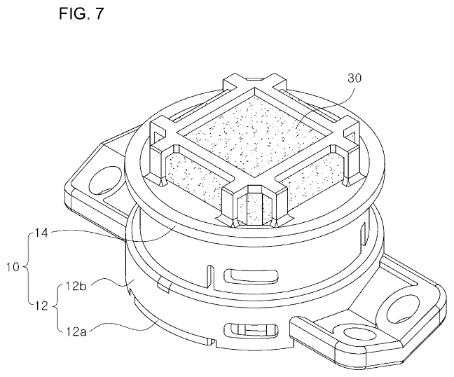

FIGS. 6 to 8 are views showing a photocatalyst device according to a second embodiment of the present invention.

The photocatalyst device according to the second embodiment has the substantially the same configuration as that of the photocatalyst device according to the first embodiment except for the light processing part 40. Hereinafter, the description will focus on the light processing unit 40, which has a structure different from that of the photocatalyst device according to the first embodiment.

The light processing part 40 includes a fly eye lens 60 installed between the light exit portions of the light sources 24 and the catalyst part 30.

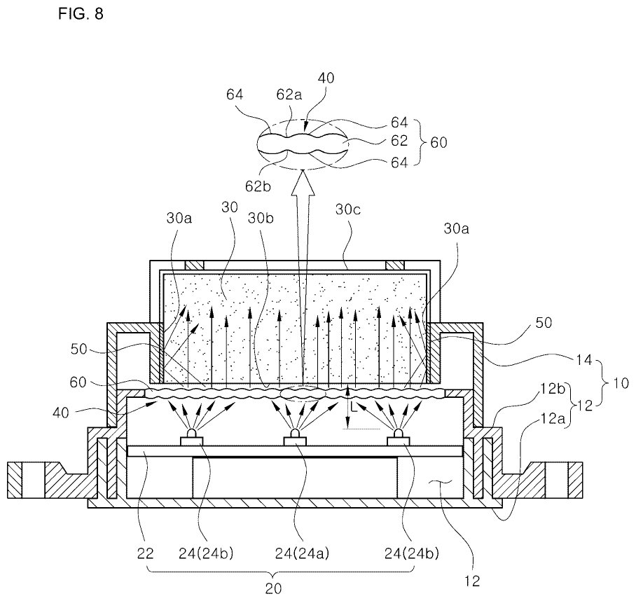

The fly eye lens 60 is made by attaching several fine lenses like the compound eyes of an insect. As shown in FIG. 8, the fly eye lens 60 includes a flat lens body sheet 62 and a plurality of lens cells 64 formed at intervals on the surface of the lens body sheet 62.

The lens body sheet 62 is a flat plate having a predetermined thickness, is made of transparent glass or transparent synthetic resin, and is installed so as to correspond to the catalyst part 30. In particular, the lens body sheet 62 is installed at a predetermined gap from the catalyst part 30.

The lens body sheet 62 corresponds to the catalyst part 30, and has an area equal to or larger than the area of the catalyst part 30.

The lens cells 64 are composed of convex lenses, and are formed continuously in rows and columns at regular intervals on the surface of the lens body sheet 62.

The lens cells 64 are formed on the front surface portion of the lens body sheet 62, and are preferably formed on one surface 62a of the lens body sheet 62 corresponding to the catalyst part 30 and the other surface 62b of the lens body sheet 62 corresponding to the light sources 24.

In some cases, the lens cells 64 may be formed only on one surface 62a of the lens body sheet 62 corresponding to the catalyst part 30.

These lens cells 64 serve to increase the homogeneity of visible light by repeatedly overlapping the visible light emitted from the light sources 24.

In particular, by increasing the homogeneity of visible light emitted from the light sources 24, the amount and irradiation range of visible light to the catalyst part 30 can be increased.

As a result, under condition that the entire catalyst part 30 can be irradiated, it is possible to shorten the separation distance L between the catalyst part 30 and the light sources 24. As a result, it is possible to reduce the separation distance L between the catalyst part 30 and the light sources 24 while sufficiently securing visible light irradiation to the entire catalyst part 30.

Accordingly, the package can be downsized without deteriorating the photocatalytic reaction in the catalyst part 30, which makes it possible to miniaturize and slim the device.

In addition, the lens cells 64 increase the homogeneity of visible light emitted from the light sources 24, thereby improving the straight movement of visible light to the catalyst part 30.

Therefore, the visible light irradiation depth of the light sources 24 with respect to the catalyst part 30 can be increased, which allows the visible light of the light sources 24 to reach the inside of the catalyst part 30 and the portion opposite to the light sources 24.

In particular, by improving the straight movement of visible light, the visible light of the light sources 24 can be irradiated to the inside of the catalyst part 30 and the portion opposite to the light sources 24, despite the impediment of diffusion of visible light by the metal foam substrate of the catalyst part 30.

Thus, sufficient visible light can be irradiated to the entire catalyst part 30, regardless of the locations of individual portions such as the front surface portion, the back surface portion, the inner portion and the outer portion of the catalyst part 30. As a result, the photocatalytic reactivity in the catalyst part 30 can be improved by increasing the amount of visible light irradiated to the entire catalyst part 30.

Accordingly, the air purification efficiency in the passenger room can be significantly improved by increasing the amount of superoxide radicals generated in the catalyst part 30.

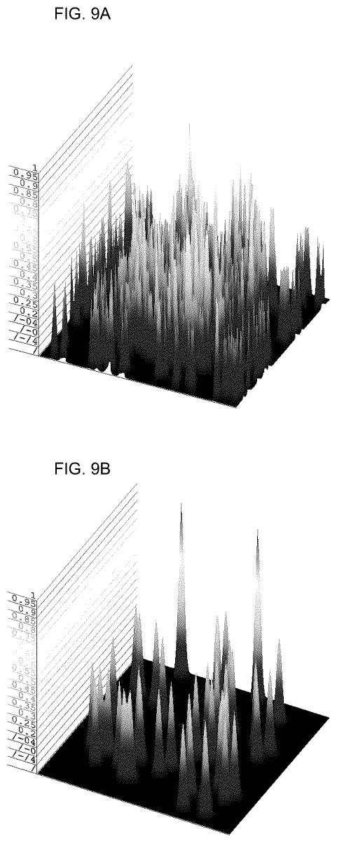

Referring to the graphs of FIGS. 9A and 9B, if the amount of visible light absorbed into the catalyst part 30 after being processed by the fly eye lens 60 in the present invention (see FIG. 9A) is compared with the amount of visible light absorbed directly into the catalyst part 30 from the light sources in the prior art (see FIG. 9B), it can be noted that the amount of visible light absorbed into the catalyst part 30 after being processed by the fly eye lens 60 in the present invention is significantly larger than the amount of visible light absorbed directly into the catalyst part 30 from the light sources in the prior art.

Thus, according to the photocatalyst device of the present invention, it is possible to downsize the package, thereby miniaturizing and slimming the device. In addition, it is possible to significantly increase the amount of visible light irradiated to the catalyst part 30, thereby improving the catalytic reactivity in the catalyst part 30. As a result, a large amount of superoxide radicals is generated, making it possible to increase the purification efficiency of the air blown into the passenger room.

According to the photocatalyst device of the present invention, visible light is irradiated to the catalyst part 30 by the plurality of light sources 24. Therefore, it is possible to significantly increase the amount of visible light irradiated to the catalyst part 30.

In addition, since the amount of visible light irradiated to the catalyst part 30 can be significantly increased, it is possible to enhance the photocatalytic reactivity in the catalyst part 30, thereby generating a large amount of superoxide radicals. As a result, it is possible to improve the purification efficiency for the air blown into the passenger room and improve the cleanliness of the air in the passenger room.

In addition, since the visible light irradiated from the light source part 20 to the catalyst part 30 is diffused through the light diffusion part 42, it is possible to significantly expand the visible light irradiation range of the light sources 24 with respect to the catalyst part 30.

In addition, since the visible light irradiation range of the light sources 24 with respect to the catalyst part 30 can be significantly expanded, it is possible to shorten the separation distance L between the catalyst part 30 and the light sources 24 under condition that the entire catalyst part 30 can be irradiated. As a result, it is possible to reduce the separation distance L between the catalyst part 30 and the light sources 24 while sufficiently securing the visible light irradiation to the entire catalyst part 30.

In addition, since the separation distance L between the catalyst part 30 and the light sources 24 can be reduced while sufficiently securing the visible light irradiation to the entire catalyst part 30, it is possible to downsize the package without deteriorating the photocatalytic reaction in the catalyst part 30, thereby miniaturizing and sliming the device.

In addition, since the straight movement of visible light irradiated from the light source part 20 to the catalyst part 30 can be improved through the light straightening part 44, it is possible to allow the visible light from the light sources 24 to reach the inside of the catalyst part 30 and the portion opposite to the light sources 24.

In addition, since the visible light from the light sources 24 can be allowed to reach the inside of the catalyst part 30 and the portion opposite to the light sources 24, it is possible to allow sufficient visible light to be irradiated to the entire catalyst part 30 regardless of the locations. As a result, it is possible to enhance the photocatalytic reactivity in the catalyst part 30 and improve the air purification efficiency in the passenger room.

In addition, since the visible light passing through the side portion 30a of the catalyst part 30 is reflected back to the inside of the catalyst part 30 through the reflection member 50, it is possible to increase the amount of visible light irradiated to the catalyst part 30. As a result, it is possible to enhance the photocatalytic reactivity in the catalyst part 30 and improve the air purification efficiency in the passenger room.

While the preferred embodiments of the present invention have been described above, the present invention is not limited to the above-described embodiments. Various modifications and changes may be made without departing from the scope and spirit of the present invention defined in the claims.

1. A photocatalyst device for generating photocatalytic reaction and emitting superoxide radicals into an internal flow path of an air conditioning case to purify air blown into a passenger room of a vehicle along the internal flow path, the photocatalyst device comprising:
a photocatalyst body;
a light source part installed on the photocatalyst body;
a catalyst part installed on the photocatalyst body so as to be spaced apart from the light source part and configured to cause a photocatalytic reaction by light emitted from the light source part; and
a light processing part configured to process the light emitted from the light source part so as to increase an irradiance rate of the light of the light source part with respect to the catalyst part.

2. The photocatalyst device of claim 1, wherein the light processing part includes a light diffusion part configured to diffuse the light emitted from the light source part to the catalyst part so as to expand a light irradiation range of the light source part with respect to the catalyst part, and a light straightening part configured to improve straight movement of the light irradiated from the light source part to the catalyst part so as to increase the depth of light irradiation of the light source part with respect to the catalyst part.

3. The photocatalyst device of claim 2, wherein the light source part includes a plurality of light sources which are LED lamps.

4. The photocatalyst device of claim 3, wherein the light sources include a central light source arranged at center and peripheral light sources arranged around the central light source at predetermined intervals.

5. The photocatalyst device of claim 4, wherein the light diffusion part includes light diffusion lenses installed on light exit portions of one or more light sources, and the light diffusion lens are configured to diffuse the light emitted from the light sources toward the catalyst part.

6. The photocatalyst device of claim 5, wherein each of the light diffusion lenses is a plano-concave lens installed so that a planar portion thereof faces the light sources and a concave portion thereof faces the catalyst part.

7. The photocatalyst device of claim 6, wherein the light diffusion lenses are installed to correspond to the peripheral light sources arranged around the central light source, and are configured to diffuse the light emitted from the peripheral light sources toward the catalyst part.

8. The photocatalyst device of claim 6, wherein the number of the light diffusion lenses is adjusted depending on the area of the catalyst part.

9. The photocatalyst device of claim 8, wherein the number of the light diffusion lenses is determined by equation (2) represented below:
number of light diffusion lenses (N)={π×(width of catalyst part (W)/2)²}/π×(separation distance between catalyst part and light source (L)×tan(90−light irradiation angle of light source enlarged by light diffusion lens/2))² (2)

10. The photocatalyst device of claim 2, wherein the light straightening part includes a light concentrating lens installed on a light exit portion of at least one of the light sources, and the light concentrating lens is configured to concentrate the light emitted from the light sources toward the catalyst part to improve the straight movement of the light toward the catalyst part.

11. The photocatalyst device of claim 10, wherein the light concentrating lens is a plano-convex lens installed so that a planar portion thereof faces the light sources and a convex portion thereof faces the catalyst part.

12. The photocatalyst device of claim 11, wherein the light concentrating lens is installed to correspond to the central light source among the plurality of light sources, and is configured to concentrate the light emitted from the central light source on the catalyst part.

13. The photocatalyst device of claim 12, wherein the light concentrating lens is installed on the light source side only under a condition that the thickness of the catalyst part exceeds a preset thickness.

14. The photocatalyst device of claim 1, wherein the light processing part includes a fly eye lens installed between the light exist portion of the light source part and the catalyst part to improve homogeneity of the light irradiated from the light source part to the catalyst part.

15. The photocatalyst device of claim 14, wherein the fly eye lens includes a lens body sheet installed between the light source part and the catalyst part, and a plurality of lens cells formed at intervals on the surface of the lens body sheet to improve light homogeneity by repeatedly overlapping the light emitted from the light source part to the catalyst part.

16. The photocatalyst device of claim 15, wherein the lens cells are formed on one surface of the lens body sheet corresponding to the catalyst part and the other surface of the lens body sheet corresponding to the light source part.

17. The photocatalyst device of claim 15, wherein the lens cells are formed on one surface of the lens body sheet corresponding to the catalyst part.

18. The photocatalyst device of claim 15, wherein the fly eye lens is configured such that the area of the fly eye lens corresponding to the catalyst part is equal to or larger than the area of the catalyst part.

19. The photocatalyst device of claim 1 further comprising:
reflection members configured to reflect the light inside the catalyst part transmitted outward through a side portion of the catalyst part toward the catalyst part.

20. The photocatalyst device of claim 19, wherein the reflection members are plates coated with a light reflecting material and are installed on the side portion of the catalyst part except a rear portion on which the visible light emitted from the light source part is incident and a front portion from which superoxide radicals are emitted.

21. The photocatalyst device of claim 20, wherein the reflection member is installed at regular intervals on the side portion of the catalyst part.

22. The photocatalyst device of claim 1 wherein the light source part is configured to irradiate visible light to the catalyst part, and the light processing part is configured to process the visible light irradiated from the light source part.
